(19) United States
(12) Patent Application Publication
(10) Pub. No.: US 2025/0359835 A1
(43) Pub. Date: Nov. 27, 2025

(54) COMPUTER-IMPLEMENTED METHOD AND EVALUATION FACILITY FOR EVALUATING A SET OF PROJECTION IMAGES, COMPUTER PROGRAM, AND ELECTRONICALLY READABLE DATA CARRIER

(71) Applicant: Siemens Healthineers AG, Forchheim (DE)

(72) Inventors: Michael Manhart, Fürth (DE); Stanislav Tashenov, Heroldsbach (DE)

(21) Appl. No.: 19/210,138

(22) Filed: May 16, 2025

(30) Foreign Application Priority Data
May 21, 2024 (DE) 10 2024 204 657.4

(51) Int. Cl. A61B 6/50; A61B 6/00; G06T 5/40; G06T 5/50; G06T 7/30; G06V 10/74

(52) U.S. Cl. CPC A61B 6/504; A61B 6/481; A61B 6/5264; G06T 5/40; G06T 5/50; G06T 7/30; G06V 10/761; G06T 2207/20224; G06T 2207/30168

(57) ABSTRACT

A computer-implemented method for evaluating a set of projection images of an X-ray facility during maskless angiography in an examination region with a blood vessel structure includes: providing the set of projection images showing a passage of a contrast agent through the blood vessel structure in a covered passage time; selecting a reference image from the set of projection images; determining a weight for each projection image, wherein the weight for the projection images that do not correspond to the reference image is determined depending on a similarity of the projection image to the reference image and the weight for the reference image is determined not lower than the maximum of the weights for the other projection images; and ascertaining at least one evaluation image from the projection images, wherein the projection images are included in the ascertaining according to the determined weights.

The present patent document claims the benefit of German Patent Application No. 10 2024 204 657.4, filed May 21, 2024, which is hereby incorporated by reference in its entirety.

### TECHNICAL FIELD

The disclosure relates to a computer-implemented method and an evaluation facility for evaluating a set of projection images of an X-ray facility during maskless angiography in an examination region with a blood vessel structure. The disclosure also relates to a computer program and an electronically readable data carrier.

### BACKGROUND

Accurate visualization of blood vessel structures and, in some cases, quantification of their static parameters and flow parameters is important for a variety of medical applications. In the case of X-ray imaging, angiographic imaging for depicting such blood vessel structures, (e.g., vascular trees), may use a contrast agent introduced into the patient's vasculature and then transported through the blood vessel structure. Digital subtraction angiography has been proposed to be able to obtain evaluation images of only the blood vessel structure from a set of projection images, e.g., two-dimensional X-ray images. In this method, initially at least one mask image is recorded without contrast agent showing the anatomical background and subtracted from the fill images with contrast agent. 3D DSA has also been proposed. However, recording mask images has some disadvantages, for example, noise amplification, additional dose, and possible patient motion between recording with and without contrast agent.

So-called maskless angiography, also known as kinetic or temporal-dynamic imaging, utilizes the temporal dynamics provided by the administered contrast agent to derive evaluation images of the vascular structure without an anatomical background. Since these approaches do not require subtraction of a mask image, a higher signal-to-noise ratio may be achieved, the radiation dose may be reduced, and less contrast agent may be used. Examples of maskless angiography, which therefore derives evaluation images directly from a set of projection images without a dedicated mask image, include digital variance angiography (DVA) and virtual mask angiography (VMA).

DVA is, for example, described in an article by Viktor Imre Óriás et al., “Digital variance angiography as a paradigm shift in carbon dioxide angiography,”

Investigative Radiology 54.7 (2019), pages 428-436, with further references. This article proposes recording a plurality of underexposed projection images and deriving evaluation images by statistical analysis, i.e., statistical images with image values containing, for example, standard deviation, variance, and other time-derived parameters of X-ray attenuation for each pixel. Statistical images that use standard deviation and variance as statistical values and hence image values contain functional motion-related information that enables improved visualization of blood vessel structures. A DVA method is also described in EP 2 628 146 A2.

DE 10 2022 209 890 A1 describes a VMA-method, specifically a method for generating a virtual mask image. A plurality of projection images of an object is captured by an angiography apparatus. For at least some of all pixel positions, an extreme pixel value of the pixels of the plurality of projection images in the respective pixel position is ascertained in each case, in particular by maximum intensity projection (MIP) with iodine as the contrast agent over time. Finally, the virtual mask image is created from the extreme pixel values at the respective pixel positions. At each pixel position, the virtual mask image consequently has a respective absolute or statistically formed minimum or maximum value of some or all pixel values at this respective pixel position. It may be provided that a noise-reduced virtual mask image is obtained by adding together at least two (optionally all) of the plurality of images, including the virtual mask image, pixelwise with a basic weighting that emphasizes the extreme pixel value more strongly.

However, the problem with such approaches is that patient motion also introduces temporal dynamics and may cause artifacts in the resulting evaluation images. Currently, there are no known specific procedures for reducing patient motion for maskless angiography, in particular DVA and VMA. Known motion correction methods based on 2D-2D registration of the projection images to one another may be used. However, such two-dimensional approaches have the disadvantage that they have limitations regarding motion in a plurality of slices and non-rigid 3D motion. In other words, motion patterns that do not correspond to single-slice translational motion with the image plane result in residual motion artifacts.

### SUMMARY AND DESCRIPTION

The disclosure is therefore based on the object of providing a way of improving the image quality of evaluation images in maskless angiography, in particular with regard to patient motion.

To achieve this object, a computer-implemented method, an evaluation facility, a computer program, and an electronically readable data carrier are provided. The scope of the present disclosure is defined solely by the appended claims and is not affected to any degree by the statements within this summary. The present embodiments may obviate one or more of the drawbacks or limitations in the related art.

According to the disclosure, a computer-implemented method is provided for evaluating a set of projection images of an X-ray facility during maskless angiography in an examination region with a blood vessel structure. The method includes providing the recorded set of projection images showing the passage of a contrast agent through the blood vessel structure in a covered passage time in a number of projection images. The method further includes selecting a reference image from the projection images. The method further includes determining a weight for each projection image, wherein the weight for the projection images that do not correspond to the reference image is determined depending on a similarity of the projection image to the reference image and the weight for the reference image is determined not lower than the maximum of the weights for the other projection images. The method further includes ascertaining at least one evaluation image from the projection images, wherein the projection images are included in the ascertaining according to the determined weights.

Therefore, at the beginning of the method, a plurality of projection images, in particular radiographs, of a set of projection images, (e.g., 20 to 40 projection images), of the examination region previously recorded is provided. In this case, in particular a contrast agent was administered to the patient, so that projection images recorded over a passage time of the contrast agent show the temporal dynamics of the contrast agent inflow, filling, and outflow over the passage time. However, the projection images may also show patient motion in the examination region. The projection images are two-dimensional X-ray images that were recorded by an X-ray facility, in particular an angiography facility.

A reference image symbolizing a reference motion state is now selected from this set of projection images. As will be explained in more detail, the reference image is in particular a projection image in which the vascular structure is clearly visible, since the aim is to extract the reference image as accurately as possible and therefore provide a good reference. First, weights are now ascertained for the other remaining images. These weights indicate how well the respective other projection image matches the reference image, for example, as a similarity of the motion states. If these weights, which may also be referred to as motion weights, are then taken into account when ascertaining the evaluation image, in particular components of similar motion states are thus emphasized and hence artifacts due to differences in the motion states are suppressed.

This is based on the idea that, although both the contrast agent flow and the patient motion are temporal dynamic effects that are reflected in the projection images because they affect the intensity, the contrast agent changes are weaker effects that are hence overshadowed by actual patient motion, which result in hard edge shifts. In other words, the similarity of the images, in particular with regard to the motion states, dominates the actual patient motion, so that the weights actually indicate which motion state deviations are present. Nevertheless, in order to reinforce this dominance of the actual motion (as opposed to changes in the contrast agent concentration), image points, i.e., pixels, of the projection images located outside a vascular mask, which indicates where the vascular structure is mapped in the projection images, may be used to determine weights. A vascular mask, which may also include a tolerance range around an actually determined vessel course for better buffering of motion, may be ascertained using a so-called vesselness filter, as discussed in more detail below.

The present disclosure thus introduces a weighting scheme for reducing motion artifacts in maskless angiography imaging. In this way, the overall influence of temporal dynamics caused by patient motion may be significantly reduced. In particular, motion artifacts may be reduced, thereby significantly increasing image quality.

Herein, weighting schemes for reducing motion artifacts in conventional digital subtraction angiography have already been proposed for a large number of recorded mask images. The present disclosure allows the advantages of such motion artifact reduction to be combined with those of maskless angiography, in particular a higher signal-to-noise ratio and reduced dose. Herein, in particular, the recognition of the dominance of actual patient motion with regard to temporal dynamics when considering the similarity of projection images is a key factor that enables the transfer of the technique from non-contrast combination techniques for ascertaining mask images.

Depending upon the specific approach used, (e.g., VMA or DVA), it is possible to ascertain different types of evaluation images. It is particularly expedient if the evaluation image or at least one of the evaluation images is a blood vessel structure image with at least reduced background features. In principle, both VMA and DVA make it possible to obtain evaluation images comparable to digital subtraction angiography, from which the anatomical (and possibly other) background is at least substantially subtracted, i.e., wherein the images substantially only show the vascular structure or the contrast agent contained therein and/or its dynamics. The quality of other types of evaluation images, (e.g., those that reproduce the dynamics by color coding), may also be significantly improved by the procedure described herein.

There is a large number of conceivable options for ascertaining the reference image. For example, expedient developments of the present disclosure provide that the reference image is the projection image of maximum contrast agent filling, in particular ascertained from an average value and/or histogram of the image values of the projection images and/or on the basis of user input, and/or the reference image is ascertained by applying a trained selection function. Maximum contrast agent filling means that as much of the vascular structure as possible is visible in the reference image. Since angiography aims to depict the vascular structure, it is particularly expedient to use a reference image in which this may be identified particularly completely, which is the case with maximum contrast agent filling. While the reference image may be selected on the basis of user input, automatic selection by a selection function that may be trained, i.e., is based on machine learning, is also possible. In the case of automatic selection by a selection function, the selection function may evaluate average values and/or histograms of the image values and/or include a so-called vesselness filter, which provides information about which feature in the image entails vessels (since these have a particularly high “vesselness”). For example, it is possible to find the projection image with the most vessels. This corresponds to the maximum contrast agent filling. If artificial intelligence, i.e., a trained selection function, is used, it may implement the vesselness filter, but may also be trained in other ways, for example, based on user selection from various projection image sets.

With regard to possible vesselness filters, reference is made by way of example to the article by Alejandro F. Frangi et al, “Multiscale Vessel Enhancement Filtering,” Medical Image Computing and Computer-Assisted Intervention—MICCAI'98, MICCAI 1998. Lecture Notes in Computer Science, Vol. 1496, pages 130-137, in which reference is also made to other conceivable approaches in addition to the multiscale approach described therein.

In a particularly advantageous embodiment, the weights may be determined in an optimization process for minimizing a deviation function for deviation images calculated by subtracting from the reference image a sum of the other projection images that do not correspond to the reference image projection images ascertained with a test set of weights. In this case, an attempt is made to reproduce the reference image from the other projection images, wherein those that have the greatest similarities to the reference image contribute the most. This provides a natural way of describing the similarity in relation to the motion state, which is also easy to implement algorithmically due to the many known techniques for solving optimization problems. High-quality weights may be derived.

Herein, the deviation function may be minimized in the form of a convex quadratic program of a dimension corresponding to the projection image number minus one. It is proposed to convert the optimization problem into a quadratic program, thereby enabling a reduction in computational effort by a factor of 100 to 1000 by reducing the dimensionality to the projection image number minus one. At the same time, it is still possible to ascertain optimal weights in a robust manner. Therefore, this provides a highly efficient way of solving the weighting problem. The optimization problem is converted into a low-dimensional quadratic program, which may be solved with less computational effort. There are already freely available approaches for quadratic programs, so that the effort required for specific implementation is significantly reduced. In particular, the creation of a general algorithm for solving nonlinearly constrained problems that is efficient in terms of computing time is avoided. Experiments have shown that the approach enables the weights of the other projection images to be ascertained in a robust manner in a very low real-time-compatible calculation mode, for example, on laptop computers as computing facilities.

In certain examples, an L2 norm, (e.g., a weighted L2 norm), may be used as the deviation function. This L2 norm of the respective deviation images for the test sets is to be minimized in the optimization process. Expediently, the quadratic program may be formulated by expanding the subtraction terms for the deviation images and separating the quadratic and linear terms in a vector of weights.

In the following, let P be the number of image points in the projection images D, the reference image R, and the deviation image, and let N be the number of projection images (projection image number), so that N−1 other projection images Dᵢ exist in addition to the reference image R. Furthermore, let d, r and a be the vectorized projection, reference and deviation images (in each case with P real-valued entries, the image values). Let the weight for the i-th other projection image Dᵢ be aᵢ wherein each weight lies in an interval of 0 to 1 and the sum of the weights of the weights of the other projection images is 1. The optimization problem may then be written as:

$$α = arg min {( r - {\sum }_{i} {α}_{i} {d}_{i} )}^{T} ( r - {\sum }_{i} {α}_{i} {d}_{i} ) s . t . {\sum }_{i} {α}_{i} = 10 \leq {α}_{i} \leq 1 \qquad ( 1 )$$

Now, let M be a P×(N−1) matrix containing the vectorized other projection images dᵢ as columns, M=(d₁ . . . d_{N-1}), and α the vector of the mask weight αᵢ with (N−1) entries. This produces:

$$α = arg min {( M α - r )}^{T} ( M α - r ) s . t . {\sum }_{i} {α}_{i} = 10 \leq {α}_{i} \leq 1 \qquad ( 2 )$$

$$α = arg min {α}^{T} {M}^{T} M α - 2 {r}^{T} M α s . t . {\sum }_{i} {α}_{i} = 10 \leq {α}_{i} \leq 1 \qquad ( 3 )$$

and, with:

$$Q = {M}^{T} M ∈ {N - 1 \times N - 1}^{} and c = {( - 2 {r}^{T} M )}^{T} ∈ {N - 1}^{} \qquad ( 4 )$$

Finally, the quadratic program:

$$α = arg min {α}^{T} Q α + {c}^{T} α s . t . {\sum }_{i} {α}_{i} = 10 \leq {α}_{i} \leq 1. \qquad ( 5 )$$

This quadratic program only includes matrices and vectors of dimensionality N−1, i.e., the number of other projection images. There is no need to evaluate the original projection images of the dimensionality P during the optimization process, wherein N−1 is much smaller than P, i.e., N−1<<P. The complete projection images only have to be processed if Q and c are precalculated. By construction, Q is positive (semi-)definite, so that the optimization problem is convex.

As equation (4) shows, both Q and c may be expressed as scalar products of the vectorized projection images dᵢ, r of the set of projection images:

$$Q ( r , c ) = {d}_{r}^{T} {d}_{c} mit r , c = 1 … N - 1 \qquad ( 6 )$$

for the quadratic term, and:

$$c ( r ) = - {d}_{r}^{T} r mit r = 1 … N - 1 \qquad ( 7 )$$

for the linear term.

In other words, to form the square matrix of the quadratic term of the deviation function in the form of the quadratic program, scalar products of the vectorized other projection images may be formed with one another and, to form the vector of the linear term of the deviation function in the form of the quadratic program, scalar products of the vectorized reference image are formed with the vectorized other projection images.

For such convex quadratic optimization problems, special algorithms already exist in the prior art that may be executed faster than algorithms for constrained nonlinear optimization. In an embodiment, the optimization process may be carried out by a Goldfarb-Idnani method. In particular it is possible to use a “Goldfarb-Idnani active-set dual method,” such as is, for example, available in toolboxes called QuadProg. The Goldfarb-Idnani-method is fundamentally described in an article by D. Goldfarb and A. Idnani, “A numerically stable dual method for solving strictly convex quadratic programs,” Mathematical Programming 27.1 (1983): 1-33. Furthermore, reference is made to the article by N. L. Boland, “A dual-active-set algorithm for positive semi-definite quadratic programming,” Mathematical Programming 78 (1996): 1-27 for positive semi-definite problems.

While an optimization process may be used, e.g., with respect to a quadratic program, the weights may be determined in another way. For example, the weights may be at least partially determined in dependence on a correlation measure and/or comparison measure of the other projection images with respect to the reference image. In this case, it is possible to use known correlation measures and/or comparison measures based on the comparison of different images. For example, it is then conceivable to ascertain the weights by normalizing the corresponding measures to one.

Finally, it is also necessary to determine a weight for the reference image itself. Since the reference image is naturally similar, the disclosure nevertheless does not aim to achieve excessively high values for the weighting, since, despite the weighting, the image contents of many projection images may be taken into account to a significant extent when ascertaining the evaluation image. For example, a particularly advantageous development provides that the weight for the reference image is determined as the maximum of the weights for the other projection images. In the formulation given above, the weights wᵢ for all projection images may therefore be obtained as

$${w}_{i} = \{ \begin{matrix}{α}_{i} & if i < {N}_{R} \\ max ( α ) & if i = {N}_{R} \\ {α}_{i - 1} & if i > {N}_{R}\end{matrix} , \qquad ( 8 )$$

wherein N_{R} is the index of the reference image.

Herein, the total sum of the weights, i.e., taking into account the weight of the reference image, does not necessarily have to be normalized to one, but this is quite conceivable and may be expedient if the sum of the weights is renormalized after adding the weight for the reference image.

Depending on how the at least one evaluation image is ascertained, the weights may be applied differently in order to allow the projection images of the set of projection images to contribute differently. In certain examples, the weights may be used in the formation of at least one weighted sum and/or in a selection of projection images for an ascertaining act when ascertaining the evaluation image, wherein in particular only projection images lying above a threshold value for the respective weight are selected. If a sum is formed, for example, in the case of pixelwise averaging or other combinations of projection images, the weights may be introduced particularly easily in the sense of a weighted sum. However, they may also be used in other cases, for example, when selecting certain projection images for an ascertaining act, in which, for example, all projection images with a weight above a threshold value area are selected. Specifically, herein, it may be provided that the threshold value may be ascertained as 0.5 divided by the projection image number, i.e., as the formula:

$${w}_{i} > \frac{μ}{N} with μ = 0.5 \qquad ( 9 )$$

With such a selection, a sufficient projection image number may be selected. However, if the threshold value results in too few images being selected, various specific approaches are conceivable.

For example, if fewer than a minimum projection image number, (e.g., 10 to 30% of the projection images), would be selected on the basis of a predetermined threshold value, either the threshold value may be reduced until the minimum projection image number is selected or the determination of the weights may be repeated, wherein a regularization technique is used to broaden the distribution of the determined weights. It is therefore initially possible to lower the threshold value, for example, lowering u in equation (9), in order to provide the selection of at least the minimum projection image number.

However, a particularly advantageous alternative for this case provides for the use of a regularization technique. In this case, it is possible to use known regularization techniques, for example, Tikhonov regularization as known from linear regression or variants thereof. Specifically, in the case of minimization in the form of a quadratic program, as described above, the present disclosure may provide that, when minimizing the deviation function in the form of a convex quadratic program, the identity matrix multiplied by a regularization factor is added to the matrix of the quadratic term.

In this case, therefore, the optimization problem, see formula (5), may be written as:

$$α = argmin {α}^{T} ( Q + \tilde{λ} I ) α + {c}^{T} α s . t . {\sum }_{i} {α}_{i} = 10 \leq {α}_{i} \leq 1 \qquad ( 10 )$$

Herein, the parameter (regularization factor) \tilde{λ}∈ [symbol] ⁺ may be selected empirically or increased until the minimum number is selected.

Some specific applications, in particular with regard to specific types of evaluation images, are explained in more detail below.

For example, a digital variance angiography (DVA) statistical image may be ascertained as at least one of the at least one evaluation image, wherein the weights are applied both in the pixelwise formation of an average over time and in the pixelwise ascertaining of a statistical value used as the image value of the statistical image, in particular variance and/or standard deviation over the passage time. Such a variant of DVA may also be referred to as DWVA (“digital weighed variance angiography”). Expressed in formulas, a DWVA image may be ascertained pixelwise as:

$$DWVA = {\sum }_{i = 1}^{N} {{w}_{i} ( {D}_{i} - {M}_{w} )}^{2} mit {M}_{w} = {\sum }_{i = 1}^{N} {w}_{i} {D}_{i} \qquad ( 11 )$$

For application in VMA, the at least one of the at least one evaluation image may be ascertained using a virtual mask image, wherein the weights are used in the selection of projection images for extreme value projection over time and/or, as additional weighting to basic weighting, when ascertaining a noise-reduced virtual mask image and/or when ascertaining the evaluation image as a weighted sum of subtraction images of the projection images and the virtual mask image.

Therefore, first, when ascertaining the original mask by temporal extreme value projection, (e.g., temporal maximum intensity projection (MIP) with iodine as a contrast agent), some projection images may be excluded by selecting only those projection images with a motion phase close to the reference image, wherein the threshold value used for the selection may as described above, be 0.5 divided by the projection image number. If a noise-reduced mask is ascertained, as also described in DE 10 2022 209 890 A1, the motion weights may be used as additional weighting to multiply the basic weight in the weighted sum. Independently of ascertaining a noise-reduced mask image, a weighted sum may also be formed using the motion weights, even if the VMA evaluation image is determined as the sum of subtraction images. The inclusion of weights when using virtual mask images may also be referred to as WVMA (“weighted virtual mask angiography”).

The motion weights may also be used in other contexts, for example, with regard to evaluation images to be depicted that are intended to visualize facts, to improve image quality. For example, it may be provided that a flow information image is ascertained as at least one of the at least one evaluation image in which areas are colored, in particular pixelwise, depending on the arrival time of the contrast agent in this area, wherein, to generate the flow information image, projection images with a weight below a threshold value are excluded. Such depiction techniques may be known by the term “iFlow.” Excluding projection images that deviate in terms of the state of motion also enables better image quality to be achieved. Herein, such flow information images may also be ascertained on the basis of the DWVA images and/or WVMA images.

In an embodiment, before the determination of the weighting, a motion correction process may be performed on the projection images, in particular including a rigid or affine 2D-2D registration of the projection images to one another for ascertaining correction factors. This enables a further improvement in image quality to be achieved, since motion artifacts are already reduced in such an act and the quantity of projection images with comparable motion states is increased. Herein, it is of particular advantage if the 2D-2D registration takes place at least between the reference image and the other projection images, since then some of the motion differences between the reference image and the other projection images have already been eliminated and there is greater similarity.

In an advantageous development, a plurality of reference images may be used and the at least one evaluation image may be ascertained for each reference image, wherein, in a selection process, one of the evaluation images is selected as the final evaluation image depending on a trained assessment function and/or an image quality measure, in particular maximum vesselness, and/or user selection information. Instead of a single reference frame, weights may also be determined for a plurality of reference images. This is in particular computationally efficient when solving the optimization problem in the form of a quadratic program, since only entries in Q and c need to be replaced and the quadratic program may also be solved with little computational effort. For example, various DWVA images and/or WVMA images may be ascertained and the best one selected. This selection may be made manually by the user, but also automatically, for example, by a trained assessment function. Such a trained assessment function may work on the basis of image quality measures, such as maximum vesselness, and/or based on annotations.

In addition to the method, an evaluation facility for evaluating a set of projection images of an X-ray facility during maskless angiography in an examination region with a blood vessel structure is disclosed. The evaluation facility includes an input interface for receiving the recorded set of projection images showing the passage of a contrast agent through the blood vessel structure in a covered passage time in a number of projection images. The evaluation facility further includes a selection unit for selecting a reference image from the projection images. The evaluation facility further includes a determination unit for determining a weight for each projection image, wherein the weight for the projection images that do not correspond to the reference image is determined depending on a similarity of the projection image to the reference image and the weight for the reference image is determined not lower than the maximum of the weights for the other projection images. The evaluation facility further includes an ascertaining unit for ascertaining at least one evaluation image from the projection images, wherein the projection images are included in the ascertaining according to the determined weights.

All statements regarding the method may be transferred analogously to the evaluation facility and vice versa, so that the advantages already mentioned may also be obtained with the evaluation facility.

The evaluation facility may have at least one processor and at least one storage device. Functional units may be formed by hardware and/or software in order to perform the acts of the method described herein. In addition to the aforementioned functional units (e.g., selection unit, determination unit and ascertaining unit), it is also possible for other functional units to be provided by other acts, for example, a correction unit for motion correction by 2D-2D registration, and/or subunits, for example, an optimization subunit for minimizing the deviation function in the optimization process as a subunit of the determination unit. The evaluation facility may also have an output interface via which the at least one ascertained evaluation image may be output.

Particularly advantageously, the evaluation facility may be part of a control facility of the X-ray facility with which the set of projection images is recorded. Therefore, an X-ray facility having an evaluation facility, in particular as part of a control facility of the X-ray facility, is conceivable. In this case, the interfaces may be internal interfaces.

The X-ray facility may be an angiography facility and/or an X-ray facility with a C-arm on which an X-ray detector and an X-ray emitter are arranged opposite one another. Such C-arm X-ray facilities are frequently used in angiography laboratories and have the advantage that they enable easily adjustable different projection directions of the projection images.

A computer program may be loaded directly into a storage device of an evaluation facility and may have program means such that, when the computer program is executed, the evaluation facility is prompted to perform the acts of a method as described herein. The computer program may be stored on an electronically readable data carrier, which therefore includes control information stored thereon, which includes at least one computer program and is configured in such way that, when the data carrier is used in an evaluation facility, the latter is embodied to perform the acts of a method. The data carrier may be a non-transient data carrier, for example, a CD-ROM.

Further advantages and details of the present disclosure are apparent from the following description of exemplary embodiments and with reference to the drawings.

### BRIEF DESCRIPTION OF THE DRAWINGS

FIG. 1 depicts a flowchart of an embodiment of the method.

FIG. 2 depicts a schematic sketch of an example of an X-ray facility.

FIG. 3 depicts an example of the functional structure of a control facility of the X-ray facility.

### DETAILED DESCRIPTION

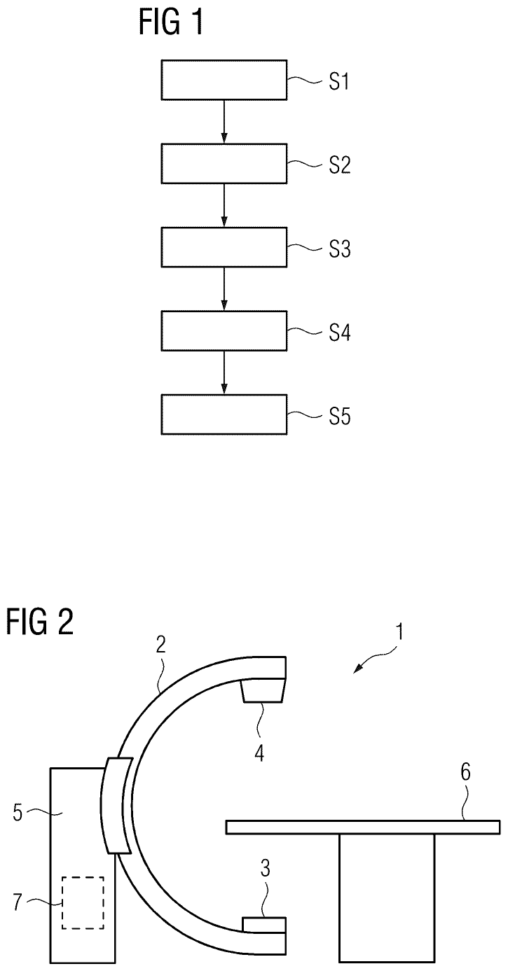

FIG. 1 shows a flowchart of an exemplary embodiment of the method. Herein, in act S1, a set of projection images is provided that includes a quantity of projection images, referred to as the projection image number, of an examination region of a patient in which a blood vessel structure has been flowed through by a contrast agent in a passage time of an angiographic imaging process. Herein, in the present case, no mask image is recorded. In other words, maskless angiography is performed. For example, there may be 20 to 40 projection images.

In act S2, a reference image is selected from the projection images in the set of projection images. Herein, in the present case, the reference image selected is the projection image with maximum contrast agent filling. This may be done manually by a user or by a selection function, in particular a trained selection function, which implements a vesselness filter and/or evaluates average intensities (image values) and/or histograms thereof, so that the projection image with the most image portions identified as vessels may be selected.

In an optional act S3, a first motion correction is performed in such a way that rigid or affine 2D-2D registration is performed between the other projection images and the reference image in order to reduce the differences in the motion state between these images and to take a first step to avoid motion artifacts. In certain examples, the correction act may be carried out before selecting the reference image, wherein the projection images are then also all shifted toward a reference motion state by the 2D-2D registration.

In act S4, weights are determined for the projection images of the set of projection images based on the similarity of the other projection images to the reference image. First, the weights for the other projection images, i.e., all projection images except the reference image, are ascertained. Herein, an optimization process is used to minimize a deviation function for deviation images, which are calculated by subtracting a sum of the other projection images that do not correspond to the reference image ascertained with a test set of weights. The test set of weights that minimizes the value of the deviation function is determined as the motion weights.

In this case, optimization is based on the quadratic program according to formula (5). For this, Q and c (formula (4)) are calculated in advance by evaluating the scalar products according to equations (6) and (7). The actual optimization is then carried out using a Goldfarb-Idnani method. For test sets of motion weights, deviation images are ascertained and evaluated by the L2 norm as a deviation measure.

After ascertaining the weights for the other projection images, a weight for the reference image is also added according to equations (8). In the present case, this is the maximum of the weights for the other projection images.

In act S5, the weights determined in act S4 are used to ascertain at least one evaluation image. Herein, the weights may be used both in a selection of projection images for an ascertaining act in such a way that only projection images with a weight above a threshold value are selected (see formula (9)) and in a summation in such a way that a weighted sum is formed.

If too few projection images are selected when using a threshold value, in particular fewer than a minimum number (e.g., 5 or 6) of projection images, act S4 may be repeated in such a way that a broader distribution of the weights results. For this purpose, a regularization term is added to the quadratic part of the optimization problem according to equation (10). This is possible since the optimization process may be carried out in a computationally efficient manner. Alternatively, it is also possible to reduce the threshold value, for example, by decreasing u, until the minimum number is reached.

In act S5, a DWVA image (see, for example, equation (11)) or a WVMA image may be ascertained as at least one of the at least one evaluation image, for example, wherein, in the latter case, an initial mask image is ascertained by extreme value projection of projection images selected on the basis of the threshold value and then a noise-reduced virtual mask image is ascertained by weighted summation of the mask image and the projection images. Herein, the weighting consists of basic weighting, which weights the mask image significantly more heavily, and motion weighting defined by the motion weight ascertained in the act as additional weighting. Finally, the WVMA image is determined by weighted summation of the respective subtraction images formed from the projection images and the noise-reduced mask image, once again using the weights from act S4.

Both these evaluation images may be used as the basis for a flow information image in which flow information is depicted in color-coded form and projection images to be used to ascertain the flow information may in turn be selected on the basis of the threshold value or a further threshold value based on the weights.

Finally, the at least one evaluation image ascertained may be output.

Although the exemplary embodiment is described for a single reference image, it is also possible to select a plurality of reference images and ascertain weights and evaluation images for each reference image. The best evaluation image of these evaluation images may be selected, for example, manually, by an assessment function and/or based on an image quality measure.

FIG. 2 shows a schematic sketch of an X-ray facility 1, which in the present case has a C-arm 2 with an X-ray emitter 3 and an X-ray detector 4 located opposite one another. The C-arm 2 is mounted on a stand 5 and is movable so that various projection geometries may be set in relation to a patient arranged on a patient couch 6.

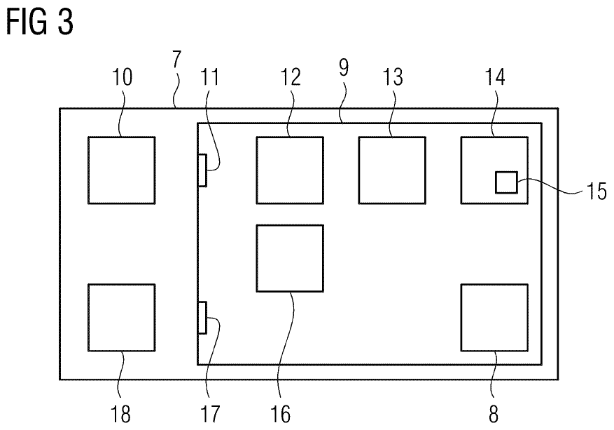

The operation of the X-ray facility 1 is controlled by a control facility 7 that includes an evaluation facility. Its functional structure is described in more detail with reference to FIG. 3. The control facility 7 includes a storage device 8, which is also used by the evaluation facility 9.

A recording unit 10 of the control facility 7 controls the recording operation, in particular also the projection images of the set of projection images and may provide this via an internal input interface 11 of the evaluation facility 9 of a selection unit 12 for selecting the reference image according to act S2. In an optional correction unit 13, motion correction may be carried out by 2D-2D registration according to the optional act S3. A determination unit 14 is embodied to determine the weights according to act S4 and accordingly also has an optimization subunit 15. In an ascertaining unit 16, it is possible to ascertain the at least one evaluation image according to act S5.

The at least one evaluation image ascertained may be provided via an output interface 17 of the evaluation facility 9 to a display unit 18 of the control facility 7 and output to a display facility of the X-ray facility 1. Ascertained evaluation images may also be stored in the storage device 8 or forwarded/held in some other way.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for evaluating a set of projection images of an X-ray facility during maskless angiography in an examination region with a blood vessel structure, the method comprising:
providing the set of projection images showing a passage of a contrast agent through the blood vessel structure in a covered passage time in a number of projection images in the set of projection images;
selecting a reference image from the set of projection images;
determining a weight for each projection image in the set of projection images, wherein weights for projection images in the set of projection images that do not correspond to the reference image are determined depending on a similarity of a respective projection image to the reference image, and wherein a weight for the reference image is determined to be not lower than a maximum of weights for the other projection images; and
ascertaining at least one evaluation image from the set of projection images using the determined weights for the projection images in the set of projection images.

2. The method of claim 1, wherein the projection image of a maximum contrast agent filling is selected as the reference image and/or the reference image is ascertained by applying a trained selection function.

3. The method of claim 2, wherein the maximum contrast agent filling is ascertained from an average value and/or histogram of image values of the projection images and/or based on user input.

4. The method of claim 1, wherein the weights are determined in an optimization process for minimizing a deviation function for deviation images calculated by subtracting, from the reference image, a sum of the other projection images that do not correspond to the reference image ascertained with a test set of weights.

5. The method of claim 4, wherein the deviation function is minimized in a form of a convex quadratic program of a dimension corresponding to the projection image number minus one.

6. The method of claim 5, wherein an L2 norm is used as the deviation function.

7. The method of claim 6, wherein the convex quadratic program is formulated by expanding subtraction terms and separating a quadratic term and linear term in a vector of weights.

8. The method of claim 7, wherein scalar products of the vectorized other projection images are formed with one another to form a square matrix of the quadratic term of the deviation function in the form of the convex quadratic program, and
wherein scalar products of the vectorized reference image are formed with the vectorized other projection images to form a vector of the linear term of the deviation function in the form of the convex quadratic program.

9. The method of claim 5, wherein the optimization process is carried out by a Goldfarb-Idnani method.

10. The method of claim 1, wherein the weights are used in a formation of at least one weighted sum and/or in a selection of projection images for an ascertaining act during the ascertaining of the evaluation image, and
wherein only projection images lying above a threshold value for the respective weight are selected.

11. The method of claim 10, wherein, when fewer than a minimum projection image number would be selected due to a predetermined threshold value, the method further comprises:
reducing the predetermined threshold value until the minimum projection image number is selected; or
repeating the determination of the weights, wherein a regularization technique is used to broaden a distribution of the determined weights.

12. The method of claim 11, wherein the minimum projection image number is in a range of 10% to 30% of the number of projection images in the set of projection images.

13. The method of claim 1, further comprising, before the determination of the weighting:
carrying out a motion correction process on the set of projection images.

14. The method of claim 13, wherein the motion correction process comprises a rigid or affine 2D-2D registration of the set of projection images to one another in order to ascertain correction factors.

15. The method of claim 1, wherein a plurality of reference images is used,
wherein the at least one evaluation image is ascertained for each reference image, and
wherein, in a selection process, one evaluation image of the evaluation images is selected as a final evaluation image depending on a trained assessment function, an image quality measure, user selection information, or a combination thereof.

16. The method of claim 15, wherein the image quality measure is a maximum vesselness.

17. An evaluation facility for evaluating a set of projection images of an X-ray facility during maskless angiography in an examination region with a blood vessel structure, the evaluation facility comprising:
an input interface configured to receive the set of projection images showing a passage of a contrast agent through the blood vessel structure in a covered passage time in a number of projection images of the set of projection images;
a selection unit configured to select a reference image from the set of projection images;
a determination unit configured to determine a weight for each projection image in the set of projection images, wherein weights for projection images in the set of projection images that do not correspond to the reference image are determined depending on a similarity of the projection image to the reference image, and wherein a weight for the reference image is determined to be not lower than a maximum of the weights for the other projection images; and
an ascertaining unit configured to ascertain at least one evaluation image from the set of projection images using determined weights for the projection images in the set of projection images.

18. A non-transitory electronically readable data carrier having a computer program that, when executed on an evaluation facility, causes the evaluation facility to:
provide a set of projection images showing a passage of a contrast agent through a blood vessel structure in a covered passage time in a number of projection images in the set of projection images;
select a reference image from the set of projection images;
determine a weight for each projection image in the set of projection images, wherein weights for projection images in the set of projection images that do not correspond to the reference image are determined depending on a similarity of a respective projection image to the reference image, and wherein a weight for the reference image is determined to be not lower than a maximum of weights for the other projection images; and
ascertain at least one evaluation image from the set of projection images using the determined weights for the projection images in the set of projection images.
